SEQUENCE LISTING (10 sequences)

SEQ ID NO: 1 - RNA, 19 nt, Artificial
  synthetic
ggcuugccagcugaauauc

SEQ ID NO: 2 - RNA, 19 nt, Artificial
  synthetic
ggguuugccugcugaauau

SEQ ID NO: 3 - RNA, 19 nt, Artificial
  synthetic
gccucaguuacgugaauac

SEQ ID NO: 4 - DNA, 21 bp, Artificial
  synthetic
aaggcttgccagctgaatatc

SEQ ID NO: 5 - DNA, 21 bp, Artificial
  synthetic
agggaagctgtacaccactaa

SEQ ID NO: 6 - DNA, 21 bp, Artificial
  synthetic
tcccgaagtctccatactaaa

SEQ ID NO: 7 - DNA, 21 bp, Artificial
  synthetic
aagggtttgcctgctgaatat

SEQ ID NO: 8 - DNA, 21 bp, Artificial
  synthetic
aagcctcagttacgtgaatac

SEQ ID NO: 9 - DNA, 19 bp, Artificial
  synthetic
ccagtaaaggctacatcat

SEQ ID NO: 10 - DNA, 21 bp, Artificial Sequence
  synthetic
guacguuacgcguaacguatt

(19) United States
(12) Patent Application Publication
(10) Pub. No.: US 2009/0215876 A1
(43) Pub. Date: Aug. 27, 2009

(54) Mitotic Progression Genes and Methods of Modulating Mitosis

(76) Inventors: Randall W. King, Newton, MA (US); Susan K. Lyman, South San Francisco, CA (US); Eunah Chung, Cambridge, MA (US); Sridaran Natesan, Ashland, MA (US); Charles B. Epstein, Brookline, MA (US)

Correspondence Address: CLARK & ELBING LLP, 101 FEDERAL STREET, BOSTON, MA, 02110, US

(21) Appl. No.: 12/223,836

(22) PCT Filed: Feb. 14, 2007
(86) PCT No.: PCT/US07/03974

§ 371 Date: Mar. 9, 2009

Related U.S. Application Data
(60) Provisional application No. 60/772,987, filed on Feb. 14, 2006.

(51) Int. Cl. A61K 31/7088; C12Q 1/48; G01N 33/53; C12Q 1/37; C12Q 1/68; C40B 30/04

(52) U.S. Cl. 514/44 R; 435/15; 435/7.1; 435/23; 435/6; 506/9

(57) ABSTRACT

The invention features methods of identifying candidate therapeutic compounds for the treatment of proliferative disorder. The invention also features methods for treating a proliferative disorder.

### BACKGROUND OF THE INVENTION

Taxotere and several other important anti-mitotic cancer drugs target tubulin proteins, but such drugs have many undesirable side effects due to tubulin's involvement in dividing, but also non-dividing cells. An increase in specificity would result from targeting only those proteins that are required during mitosis and have no role in non-dividing cellular processes. A cell-based phenotypic screen performed at Harvard Medical School led to the first identification of an anti-mitotic small molecule (monastrol) that does not target tubulin, but instead acts by inhibiting the mitotic kinesin protein Eg5. Other small molecule inhibitors of Eg5 are currently clinical trials for the treatment of cancer.

High-throughput siRNA screens provide an alternative for rapid identification of novel therapeutic targets that, if inactivated, affect cellular processes of interest. Since taxanes and other microtubule inhibitors activate the spindle checkpoint, identification of novel components of this pathway may increase the understanding of the mechanism by which taxanes induce apoptosis in cancer cells and also help in the understanding of how resistance to these drugs arises.

There is a need to identify proteins that are critical to the activation of the spindle checkpoint. Such proteins would provide targets for use in the discovery of effective cancer therapeutics with decreased side effects.

### BRIEF SUMMARY OF THE INVENTION

In one aspect, the invention features a method for identifying a candidate therapeutic compound by contacting HIPK1, HIPK2, or USP32 with the compound to be assessed. In this aspect, the biological activity of HIPK1, HIPK2, or USP32 contacting the compound is compared with the biological activity of HIPK1, HIPK2, or USP32 absent the compound. Compounds are identified that reduce the biological activity of HIPK1, HIPK2, or USP32. In this embodiment, the compound that reduces the biological activity of the HIPK1, HIPK2, or USP32 is a candidate compound for the treatment of a proliferative disease (e.g., colon cancer, breast cancer, prostate cancer, lymphoma, leukemia, melanoma, ovarian cancer, pancreatic cancer, and lung cancer).

In the forgoing aspect, the contacting can be in a cell-free mixture or a cell based mixture (e.g., a recombinant cell).

In any of the forgoing aspects the biological activity of HIPK1 or HIPK2 can be, for example, a kinase activity or binding activity.

Also in any of the forgoing aspects, the biological activity of USP32 can be, for example, a protease activity or a binding activity.

In another aspect, the invention features a method of identifying a candidate compound that ameliorates a proliferative disease. In this aspect the method includes contacting a cell (e.g., a cell contained in an animal model or a cell derived from a disease model) with a candidate compound, comparing the expression of at least one of HIPK1, HIPK2, and USP32 with the expression of the gene or genes in a cell not contacted with the candidate compound, and identifying a compound which modulates the expression of at least one of the genes.

In the forgoing aspect, the expression can be assessed by reduction in disease-specific properties.

Also in the forgoing aspect, the expression can be measured using a microarray (e.g., a nucleic acid or protein microarray).

In yet another aspect, the invention features a method of treating a proliferative disease in a subject including administering an inhibitor of HIPK1, HIPK2, or USP32 to the subject. In this aspect, the inhibitor can be an siRNA construct (e.g., siRNA constructs including the nucleic acid sequences set forth in SEQ ID NOs:1-9).

By a “compound,” “candidate compound,” or “factor” is meant a chemical, be it naturally occurring or artificially derived. Compounds may include, for example, peptides, polypeptides, synthetic organic molecules, naturally occurring organic molecules, nucleic acid molecules, and components or combinations thereof.

By “modulating” is meant either increasing (“upward modulating”) or decreasing (“downward modulating”) the biological activity of a protein in vivo or ex vivo. It is important to note that the modulation may be either direct or indirect. It will be appreciated that the degree of biological activity provided by a modulatory compound in a given assay will vary, but that one skilled in the art can determine the statistically significant change in the level of biological activity that identifies a compound that increases or decreases biological activity. By “modulation of expression” is meant a change in protein or nucleic acid level or activity in a cell, a cell extract, or a cell supernatant. For example, such a change may be due to increased or decreased RNA stability, transcription, protein degradation, or translation. Preferably, this change is at least 5%, 10%, 25%, 50%, 75%, 80%, 100%, 200%, or even 500%, or more, of the level of expression or activity under control conditions.

By “cell-free mixture” is meant experimental conditions done in vitro. These conditions include experiments conducted with cell extracts, with substantially purified cell products, and/or artificial compounds.

By “cell-based mixture” is meant an experiment conducted in the presence of live cells, dead cells, or fixed cells.

By “recombinant cell” is meant a cell engineered to contain an exogenous nucleic acid. Examples are cells engineered to express a protein of interest or cells which contain a reporter construct to detect a certain biological activity.

By “kinase activity” is meant the activity whereby an enzyme phosphorylates an amino-acid residue on a protein substrate.

By “proliferative disease” is meant a disease that is caused by or results in inappropriately high levels of cell division, inappropriately low levels of apoptosis, or both. Cancers such as lymphoma, leukemia, melanoma, ovarian cancer, breast cancer, pancreatic cancer, and lung cancer are all examples of proliferative disease.

By “HIPK1” or “homeodomain interacting protein kinase I” is meant a polypeptide with the activity (e.g., kinase activity) of human HIPK1. An exemplary Genbank accession number corresponding to the nucleic acid sequence of HIPK1 is NM_{—}52696 and an exemplary Genbank Accession number corresponding to the polypeptide sequence of HIPK1 is NP_{—}689909. By HIPK1 is also meant a polypeptide with at least 50%, 60%, 70%, 80%, 90%, 95%, or 99% percent sequence identity to the HIPK1 polypeptide. Additionally and alternatively, HIPK1 is defined as a polypeptide encoded by a nucleic acid that hybridizes under high stringency conditions to a nucleic acid of HIPK1.

By “HIPK2” or “homeodomain interacting protein kinase 2” is meant a polypeptide with the activity (e.g., kinase activity) of human HIPK2. Exemplary Genbank accession numbers corresponding to the nucleic acid sequence of HIPK2 are NM_{—}022740 and AF208291 and exemplary Genbank Accession numbers corresponding to the polypeptide sequence of HIPK2 are NP_{—}073577 and Q9H2×6. By HIPK2 is also meant a polypeptide with at least 50%, 60%, 70%, 80%, 90%, 95%, or 99% percent sequence identity to the HIPK2 polypeptide. Additionally and alternatively, HIPK2 is defined as a polypeptide encoded by a nucleic acid that hybridizes under high stringency conditions to a nucleic acid of HIPK2.

By “USP32” or “ubiquitin-specific protease 32” is meant a polypeptide with the activity (e.g., protease activity) of human USP32. An exemplary Genbank accession number corresponding to the nucleic acid sequence of USP32 is NM_{—}032582 and an exemplary Genbank Accession number corresponding to the polypeptide sequence of USP32 is NP_{—}115971. By USP32 is also meant a polypeptide with at least 50%, 60%, 70%, 80%, 90%, 95%, or 99% percent sequence identity to the USP32 polypeptide. Additionally and alternatively, USP32 is defined as a polypeptide encoded by a nucleic acid that hybridizes under high stringency conditions to a nucleic acid of USP32.

By “expression” is meant the amount of a nucleic acid or protein being produced by a cell. Changes in expression may result from changes in transcription of mRNA, translation of protein, or degradation of either nucleic acids or proteins.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

By “mitotic progression” is meant the passage of a cell through the cell cycle.

By “mitotic index” is meant the percentage of cells undergoing mitosis at any chosen time.

“Protein” or “polypeptide” or “polypeptide fragment” means any chain of more than two amino acids, regardless of post-translational modification (e.g., glycosylation or phosphorylation), constituting all or part of a naturally-occurring polypeptide or peptide, or constituting a non-naturally occurring polypeptide or peptide.

The “percent identity” of two nucleic acid or polypeptide sequences can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, Academic Press, 1987; and Sequence Analysis Primer, Gribskov, and Devereux, eds., M. Stockton Press, New York, 1991; and Carillo and Lipman, SIAM J. Applied Math. 48:1073, 1988.

Methods to determine identity are available in publicly available computer programs. Computer program methods to determine identity between two sequences include, but are not limited to, the GCG program package (Devereux et al., Nucleic Acids Research 12:387, 1984), BLASTP, BLASTN, and FASTA (Altschul et al., J. Mol. Biol. 215:403, 1990). The well known Smith Waterman algorithm may also be used to determine identity. The BLAST program is publicly available from NCBI and other sources (BLAST Manual, Altschul, et al., NCBI NLM NIH Bethesda, Md. 20894). Searches can be performed in URLs such as the following: http://www.ncbi.nlm.nih.gov/BLAST/unfinishedgenome.html; or http://www.tigr.org/cgi-bin/BlastSearch/blast.cgi. These software programs match similar sequences by assigning degrees of homology to various substitutions, deletions, and other modifications. Conservative substitutions typically include substitutions within the following groups: glycine, alanine; valine, isoleucine, leucine; aspartic acid, glutaric acid, asparagine, glutamine; serine, threonine; lysine, arginine; and phenylalanine, tyrosine.

By “hybridize” is meant pair to form a double-stranded complex containing complementary paired nucleobase sequences, or portions thereof, under various conditions of stringency. (See, e.g., Wahl. and Berger, Methods Enzymol 152:399 (1987); Kimmel, Methods Enzymol 152:507 (1987))

By “hybridizes under high stringency conditions” is meant under conditions of stringent salt concentration, stringent temperature, or in the presence of formamide. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180 (1977)); Grunstein and Hogness (Proc Natl Acad Sci USA 72:3961 (1975)); Ausubel et al. (Current Protocols in Molecular Biology, Wiley Interscience, New York (2001)); Berger and Kimmel (Guide to Molecular Cloning Techniques, Academic Press, New York, (1987)); and Sambrook et al. (Molecular Cloning: A Laboratory Manual, Cold Spring Harbor Laboratory Press, New York). Preferably, hybridization occurs under physiological conditions. Typically, complementary nucleobases hybridize via hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “small interfering RNAs (siRNAs)” is meant an isolated dsRNA molecule, preferably greater than 10 nucleotides in length, more preferably greater than 15 nucleotides in length, and most preferably 18, 19, 20, 21, 22, 23, 24, or 25 nucleotides in length that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 21 to 23 nucleotide RNA or internally (at one or more nucleotides of the RNA). Recently it has been noted that larger siRNA molecules, for example, 25 nt, 30 nt, 50 nt, or even 100 nt or more, can also be used to initiate RNAi. (See for example, Elbashir et al. (Genes & Dev., 15:188-200, 2001), Girard et al. (Nature 442:199-202 (2006), Aravin et al. (Nature 442:203-207 (2006)), Grivna et al. (Genes Dev. 20:1709-1714 (2006)), and Lau et al. (Science 313:363-367 (2006)). In a preferred embodiment, the RNA molecule contains a 3′hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNAi. As used herein “mediate RNAi” refers to the ability to distinguish or identify which RNAs are to be degraded.

By a “microarray” or “array” is meant a fixed pattern of immobilized objects on a solid surface or membrane. As used herein, the array is made up of polypeptides, cDNAs, or ESTs immobilized on the solid surface or membrane. “Microarray” and “array” are used interchangeably. Preferably, the microarray has a density of between 10 and 1,000 objects/cm².

### BRIEF DESCRIPTION OF THE FIGURES

FIG. 1A is a diagram showing the relationship of HIPK1 and HIPK2 to other kinases.

FIG. 1B is a diagram showing the protein structure of HIPK1, HIPK2, and HIPK3.

FIG. 2 is a diagram showing proteins with which HIPK1 and HIPK2 interact.

FIG. 3 are graphs showing the level of interaction of HIPK2 with the indicated proteins, IRS1, and chromosome 11 orf 66.

FIG. 4 is a table and a graph showing the level of interaction of HIPK2 with the indicate proteins showing the potential phosphorylation of the indicated substrates by HIPK2.

FIG. 5 is a flow chart showing the organization of a mitotic arrest screen.

FIG. 6 is a diagram showing an overview of the mitotic arrest screen.

FIG. 7 is a table identifying genes identified in the mitotic arrest screen which were previously known to have a role in mitotic arrest and spindle formation.

FIG. 8 is a table showing the number of genes identified as having a particular arrest phenotype.

FIG. 9 is a pair of photomicrographs comparing cells treated with taxol with cells treated with taxol that are also treated with MAD2siRNA.

FIG. 10A is a table indicating percent knockdown of HIPK2 protein by siRNA.

FIG. 10B is a western blot showing HIPK2 expression levels in cells treated with the indicated siRNA constructs.

FIG. 11 is a graph showing amount of the indicated mRNA found in cells transfected with either HIPK1 or HIPK2 siRNA at stated intervals after transfection.

FIG. 12 is a graph showing the percent cell death in cells transfected with HIPK1 and/or HIPK2.

FIG. 13A is a graph showing percent cell survival of cells transfected with HIPK1 siRNA.

FIG. 13B is a graph showing percent cell survival of cells transfected with HIPK2 siRNA.

FIG. 14A is a graph showing the percentage of cell viability in cells treated with the indicated siRNA construct.

FIG. 14B is a western blot showing the amount of HIPK2 protein present in cells treated with the indicated siRNA construct.

FIG. 15 is a graph showing fold increase in apoptosis in response to the indicated siRNA constructs.

FIG. 16 is a graph showing caspase 3 induction and percentage cell viability in cells treated with the indicated siRNA construct.

FIG. 17 is a graph showing caspase activation in cells transfected with USP32 siRNA.

FIG. 18 is a graph showing the percentage of cells transfected with USP32 siRNA in the indicated stage of cell cycle.

FIG. 19 is a graph showing the percentage of HCT116 cells transfected with HIPK2 in the indicated stage of cell cycle 24 hours after transfection.

FIG. 20 is a graph showing the percentage of HCT116 cells transfected with HIPK2 in the indicated stage of cell cycle 48 hours after transfection.

FIG. 21 is a graph showing the percentage of MDA-MB231 cells transfected with HIPK2 in the indicated stage of cell cycle 48 hours after transfection.

FIG. 22 is a graph showing percentage of cells treated with the indicated siRNA constructs which are in the indicated stage of cell cycle.

FIG. 23 is a graph showing the expression of HIPK1 in normal tissues.

FIG. 24 is a graph showing the expression of HIPK1 in various NCI-60 cell lines.

FIG. 25 is a graph showing the expression of HIPK2 in various NCl-60 cell lines.

### DETAILED DESCRIPTION OF THE INVENTION

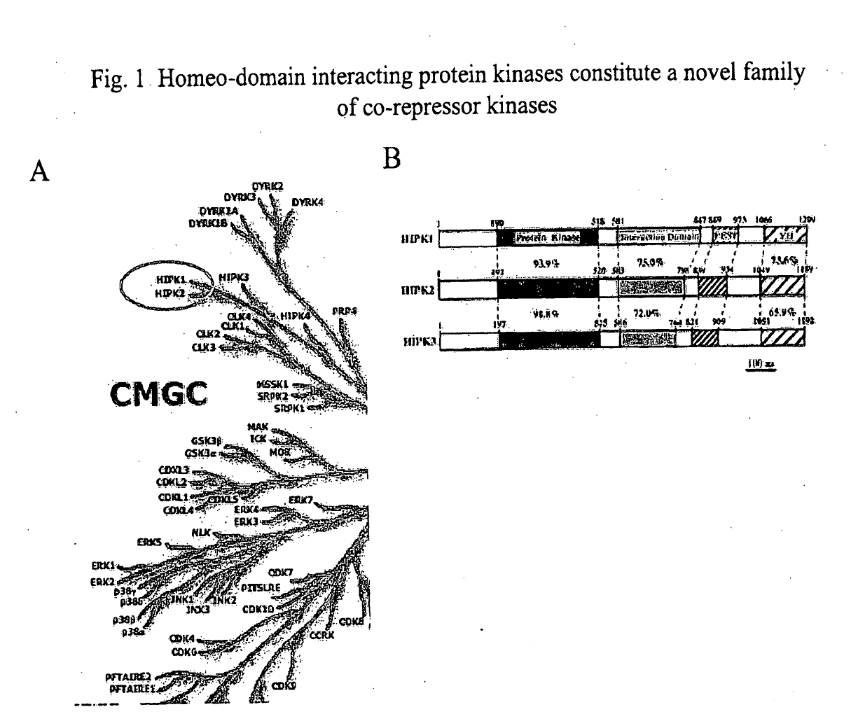

The invention features methods for identifying compounds which inhibit the activity of homeodomain interacting protein kinase 1 (HIPK1), homeodomain interacting protein kinase 2 (HIPK2), and ubiquitin-specific protease 32 (USP32). HIPK1 and HIPK2 are members of a distinct family of serine/threonine kinases (FIG. 1A). The domain structure of each protein is known and set forth in FIG. 1B.

We find that HIPK1, HIPK2, and USP32 activity are essential for the survival and division of cells derived from neoplastic tissue, but not normal cells.

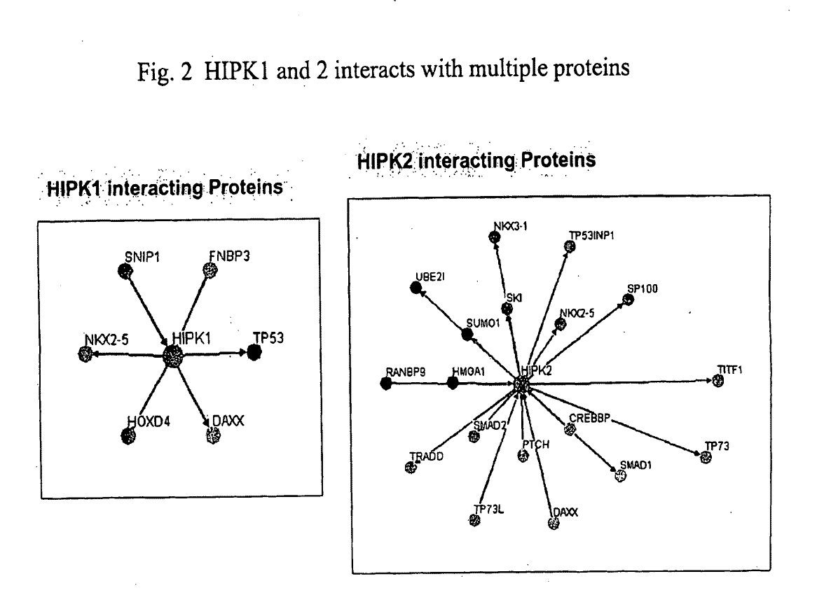

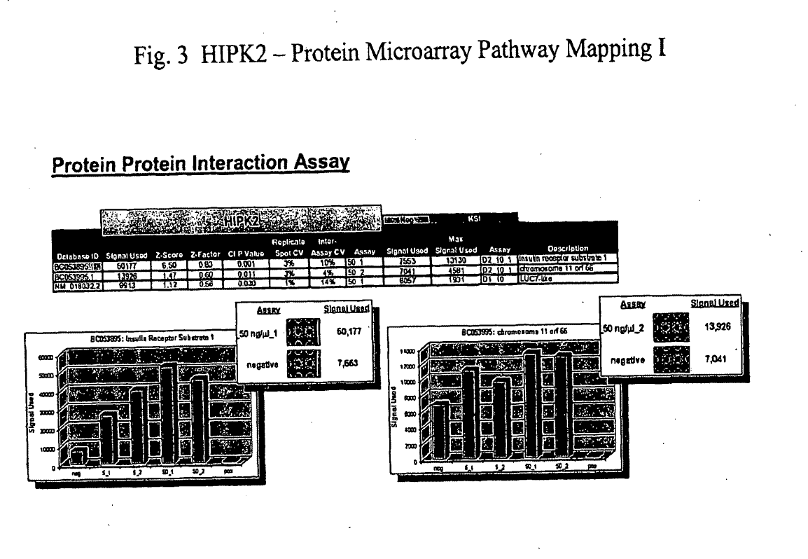

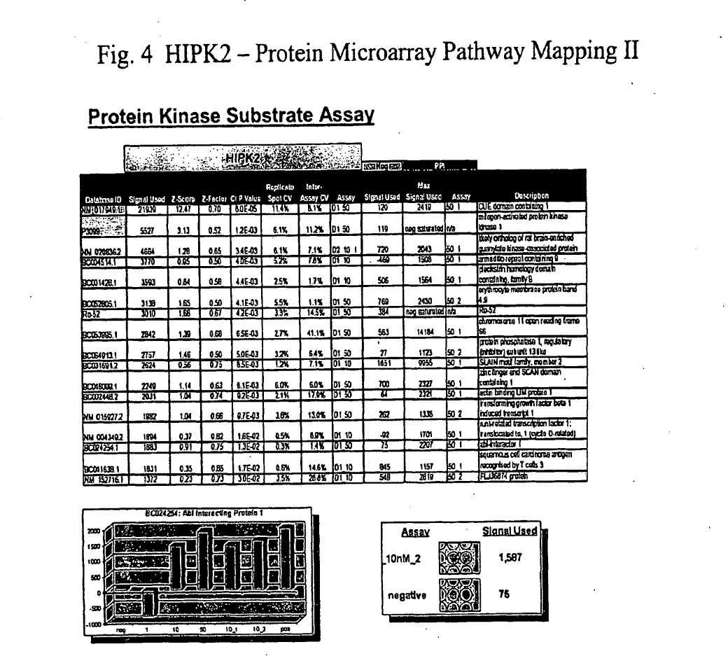

This essential activity of HIPK1, HIPK2, and USP32 may arise from their interaction with other proteins (e.g., FIGS. 2-4).

### I. Screening Methods to Identify Candidate Therapeutic Compounds

The invention provides screening methods for the identification of compounds that bind to, or modulate expression or activity of, HIPK1, HIPK2, or USP32.

### Screening Assays

Screening assays to identify compounds that modulate the expression or activity of HIPK1, HIPK2, or USP32 are carried out by standard methods. The screening methods may involve high-throughput techniques. In addition, these screening techniques may be carried out in cultured cells or in organisms such as worms, flies, yeast, or mammals. Screening in these organisms may include the use of polynucleotides homologous to HIPK1, HIPK2, or USP32.

Any number of methods is available for carrying out such screening assays. According to one approach, candidate compounds are added at varying concentrations to the culture medium of cells expressing a polynucleotide coding for HIPK1, HIPK2, or USP32. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. Gene expression can also be measured by reverse transcription followed by quantitative PCR or by hybridization to oligonucleotides (e.g., on a microarray). The level of gene expression in the presence of the candidate compound is compared to the level measured in a control culture medium lacking the candidate molecule. A compound which promotes a change in HIPK1, HIPK2, or USP32 expression is considered useful in the invention; such a molecule may be used, for example, as a therapeutic for a proliferative disorder.

While a candidate compound may be identified through modulation of any one of HIPK1, HIPK2, or USP32, particularly promising compounds would modulate both, or all of HIPK1, HIPK2, or USP32. It is well known in the art that the gene expression of a large number of genes can be measured using a nucleotide microarray. Compounds that modulate HIPK1, HIPK2, or USP32 could be identified by comparing the expression profile of HIPK1, HIPK2, or USP32 from cells treated with a candidate compound compared to a control sample.

One aspect of this invention is a microarray containing nucleic acid molecules which hybridize nucleic acids substantially identical to HIPK1, HIPK2, or USP32 or fragments thereof. Preferably, the microarray would contain nucleic acid molecules that hybridize nucleic acids substantially identical to all of HIPK1, HIPK2, or USP32 or fragments thereof. Yet another feature of the invention is the method of analyzing data from previously conducted microarray experiments, where the microarray based candidate drug screen contains HIPK1, HIPK2, or USP32, to identify candidate compounds. A feature of this aspect of the invention is that large portions of the experimentation has already been completed and is available in the art.

If desired, the effect of candidate compounds may, in the alternative, be measured at the level of polypeptide production using the same general approach and standard immunological techniques, such as Western blotting or immunoprecipitation with an antibody specific for HIPK1, HIPK2, or USP32. For example, immunoassays may be used to detect or monitor the expression of HIPK1, HIPK2, or USP32. Polyclonal or monoclonal antibodies which are capable of binding to such a polypeptide may be used in any standard immunoassay format (e.g., ELISA, Western blot, RIA assay, or protein microarray) to measure the level of HIPK1, HIPK2, or USP32 protein expression. A compound which promotes a change in the expression of HIPK1, HIPK2, or USP32 proteins is considered particularly useful. Again, such a molecule may be used, for example, as a therapeutic for a proliferative disorder.

Alternatively, or in addition, candidate compounds may be screened for those which specifically bind to and modulate the activity of HIPK1, HIPK2, or USP32. The efficacy of such a candidate compound is dependent upon its ability to interact with the polypeptide. Such an interaction can be readily assayed using any number of standard binding techniques and functional assays (e.g., those described in Ausubel et al., supra). For example, a candidate compound may be tested in vitro for interaction and binding with HIPK1, HIPK2, or USP32 and its ability to modulate its activity may be assayed by any standard assays (e.g., those described herein).

In one particular embodiment, a candidate compound that binds to HIPK1, HIPK2, or USP32 proteins may be identified using a chromatography-based technique. For example, recombinant HIPK1, HIPK2, or USP32 proteins may be purified by standard techniques from cells engineered to express HIPK1, HIPK2, or USP32 and may be immobilized on a column. A solution of candidate compounds is then passed through the column, and a compound specific for HIPK1, HIPK2, or USP32 is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat a proliferative disorder. Compounds which are identified as binding to HIPK1, HIPK2, or USP32 with an affinity constant less than or equal to 10 mM are considered particularly useful in the invention.

In another embodiment, compounds which inhibit the activity of HIPK1 and HIPK2 are identified in a kinase assay. Kinase assays are well known in the art and measure the level of kinase activity (e.g., serine/threonine kinase activity) of a particular protein. In one embodiment, kinase activity is measured by phosphorylation of a substrate (e.g., phosphorylation of p53). This phosphorylation can be determined in vitro or in a cell based system. This phosphorylation can be measured, for example, using antibodies specific for phosphorylated proteins, or by using radioactive phosphate.

Potential agonists and antagonists include organic molecules, peptides, peptide mimetics, polypeptides, and antibodies that bind to HIPK1, HIPK2, or USP32, or a polynucleotide encoding HIPK1, HIPK2, or USP32, and thereby increase or decrease its activity. Potential antagonists include small molecules that bind to and occupy the binding sites of proteins of HIPK1, HIPK2, or USP32 which are known to be enzymes. Other potential antagonists include antisense molecules.

Polynucleotide sequences coding for HIPK1, HIPK2, or USP32 may also be used in the discovery and development of compounds to treat proliferative disorders. HIPK1, HIPK2, or USP32, upon expression, can be used as a target for the screening of drugs. Additionally, the polynucleotide sequences encoding the amino terminal regions of the encoded polypeptide or Shine-Delgarno or other translation facilitating sequences of the respective mRNA can be used to construct antisense sequences to control the expression of the coding sequence of interest. Polynucleotides encoding fragments of HIPK1, HIPK2, or USP32 (e.g., SEQ ID NOs:1-9) may, for example, be expressed such that RNA interference takes place, thereby reducing expression or activity of HIPK1, HIPK2, or USP32.

Additional confirmatory experiments can include measurement of impaired protein-protein interaction (e.g., interaction with the proteins set forth in FIG. 2-4), induction of mitotic arrest, and induction of checkpoint control.

Small molecules provide useful candidate therapeutics. Preferably, such molecules have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of treating a proliferative disorder are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating proliferative disorders should be employed whenever possible.

When a crude extract is found to have an activity that modulates HIPK1, HIPK2, or USP32 protein expression or activity, or a binding activity, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating a proliferative disorder. Methods of fractionation and purification of such heterogenous extracts are known in the art. If desired, compounds shown to be useful agents for the treatment of a proliferative disorder are chemically modified according to methods known in the art.

### II. Methods of Treatment

The invention also features methods of treating a proliferative disease in a patient by administering compounds that inhibit the activity of HIPK1, HIP2, or USP32. These methods also include the administration of HIPK1, HIPK2, or USP32 siRNA constructs.

RNA interference (RNAi) is a form of post-transcriptional gene silencing initiated by the introduction of double-stranded RNA (dsRNA). Short twenty-one to twenty-five nucleotide double-stranded RNAs are effective at down-regulating gene expression in nematodes (Zamore et al., Cell 101: 25-33) and in mammalian tissue culture cell lines (Elbashir et al., Nature 411:494-498, 2001, hereby incorporated by reference). The further therapeutic effectiveness of this approach in mammals was demonstrated in vivo by McCaffrey et al. (Nature 418:38-39. 2002). The nucleic acid sequence of a mammalian gene, such as HIPK1, HIPK2, or USP32, can be used to design small interfering RNAs (siRNAs) that will inactivate HIPK1, HIPK2, or USP32 target genes that have the specific 21 to 25 nucleotide RNA sequences used. siRNAs may be used, for example, as therapeutics to treat a proliferative disease.

Provided with the sequence of a mammalian gene, dsRNAs may be designed to inactivate target genes of interest and screened for effective gene silencing, as described herein. In addition to the dsRNAs disclosed herein, additional dsRNAs may be designed using standard methods.

The specific requirements and modifications of dsRNA are described in PCT application number WO 01/75164 (incorporated herein by reference). While dsRNA molecules can vary in length, it is most preferable to use siRNA molecules that are 21- to 23-nucleotide dsRNAs with characteristic 2- to 3-nucleotide 3′ overhanging ends, preferably these are (2′-deoxy)thymidine or uracil. The siRNAs typically comprise a 3′ hydroxyl group. Alternatively, single stranded siRNAs or blunt ended dsRNA are used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs e.g. substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA molecules can be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using any of the methods set forth in PCT number WO01/75164 (incorporated herein by reference) or using standard procedures for in vitro transcription of RNA and dsRNA annealing procedures as described in Elbashir S. M. et al. (Genes & Dev., 15:198-200, 2001). siRNAs are also obtained as described in Elbashir S. M. et al., by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g. size exclusion chromatography), glycerol gradient centrifugation, and affinity purification with antibody can be used to isolate the 21 to 23 nucleotide RNAs.

Short hairpin RNAs (shRNAs) can also be used for RNAi as described in Yu et al. or Paddison et al. (Proc. Natl. Acad. Sci. USA. 99:6047-6052, 2002; Genes & Dev, 16:948-958, 2002; incorporated herein by reference). shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

Examples of such siRNA constructs are set forth below in Table 1 and the nucleic acid sequences set forth in SEQ ID NOs:4-9.

TABLE 1

siRNA constructs

Exemplary siRNA construct

| | | |
|---|---|---|
| H1PK1 | GGCUUGCCAGCUGAAUAUC | (SEQ ID NO: 1) |
| H1PK2 | GGGUUUGCCUGCUGAAUAU | (SEQ ID NO: 2) |
| USP32 | GCCUCAGUUACGUGAAUAC | (SEQ ID NO: 3) |

siRNAs that block cell proliferation or survival in tumor cells may also block differentiation of committed progenitor cells. For example, some anti-cancer compounds that induce apoptosis in tumor cell lines block differentiation of adipocytes or other committed progenitor cells. Therefore the HIPK1, HIPK2, and USP32 siRNA constructs of the invention may be used to treat metabolic diseases (e.g., obesity and type II diabetes) and neurodegenerative diseases using the therapeutic methods set forth below.

Therapeutic HIPK1, HIPK2, or USP32 RNAi

Proliferative diseases from any warm-blooded mammal may be treated using the methods of the invention. Proliferative diseases subject to such therapies include, but are not limited to, lung cancer, colon cancer, kidney cancer, bone cancer, breast cancer, prostate cancer, uterine cancer, ovarian cancer, liver cancer, pancreatic cancer, brain cancer, lymphoma, melanoma, myeloma, adenocarcinoma, thymoma, plasmacytoma, or any other neoplasm, such proliferative diseases are, preferably, characterized by having increased HIPK1, HIPK2, or USP32 expression. Warm-blooded animals include, but are not limited to, humans, cows, horses, pigs, sheep, birds, mice, rats, dogs, cats, monkeys, baboons, or other mammals.

### HIPK1, HIPK2, or USP32 Therapeutics for RNAi

The administration of HIPK1, HIPK2, or USP32 nucleic acid molecules for RNAi therapy (e.g., dsRNA, antisense RNA, or siRNA) may be provided to prevent or treat a proliferative disease. Such nucleic acid molecules may be administered directly to a tissue or neoplasm or may be provided within an expression vector, such that the nucleic acid molecule mediating the RNAi is stably expressed.

For direct administration of HIPK1, HIPK2, or USP32 nucleic acid molecules for RNAi (e.g., dsRNA, antisense RNA, or siRNA) or mixtures thereof, nucleic acid molecules are provided in a unit dose form, each dose containing a predetermined quantity of such molecules sufficient to silence a target gene in association with a pharmaceutically acceptable diluent or carrier, such as phosphate-buffered saline, to form a pharmaceutical composition. In addition, the HIPK1, HIPK2, or USP32 nucleic acid molecules for RNAi may be formulated in a solid form and redissolved or suspended prior to use. The pharmaceutical composition may, optionally, contain other chemotherapeutic agents, antibodies, antivirals, and exogenous immunomodulators.

The route of administration may be, for example, intravenous, intramuscular, subcutaneous, topical, intradermal, intraperitoneal, intrathecal, or ex vivo. Administration may also be by transmucosal or transdermal means, or the compound may be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the HIPK1, HIPK2, or USP32 nucleic acid molecule for RNAi is formulated into conventional oral administration forms, such as capsules, tablets and tonics. For topical administration, the nucleic acid molecules of the invention are formulated into ointments, salves, gels, or creams, as is generally known in the art.

In providing a mammal with the HIPK1, HIPK2, or USP32 nucleic acid molecules for RNAi, the dosage of administered nucleic acid molecules will vary depending upon such factors as the mammal's age, weight, height, sex, general medical condition, previous medical history, disease progression, tumor burden, and the like. The dose is administered as indicated. Other therapeutic drugs may be administered in conjunction with the nucleic acid molecules.

The efficacy of treatment using the nucleic acid molecules described herein may be assessed by determination of alterations in the concentration or activity of the DNA, RNA or gene product of HIPK1, HIPK2, or USP32, tumor regression, or a reduction of the pathology or symptoms associated with the neoplasm.

### Nucleic Acid Therapy

Nucleic acid therapy is another therapeutic approach for preventing or ameliorating a neoplasia related to the increased expression of an HIPK1, HIPK2, or USP32 nucleic acid molecule. Expression vectors encoding anti-sense nucleic acid molecules, dsRNAs, siRNAs, or shRNAs can be delivered to cells that overexpress an endogenous HIPK1, HIPK2, or USP32 nucleic acid molecule. Such delivery results in the sustained expression of HIPK1, HIPK2, or USP32 nucleic acid molecules for RNAi. The nucleic acid molecules must be delivered to cells in need of RNAi (e.g., neoplastic cells) in a form in which they can be taken up by the cells and so that sufficient levels of RNAi nucleic acid molecules can be produced to decrease HIPK1, HIPK2, or USP32 levels in a patient having a proliferative disease.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to express an HIPK1, HIPK2, or USP32 nucleic acid molecule capable of mediating RNAi.

Non-viral approaches can also be employed for the introduction of an RNAi therapeutic to a cell of a patient having a neoplasia. For example, a nucleic acid molecule can be introduced into a cell by administering the nucleic acid in the presence of lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acid molecules are contained within plasmid vectors and are administered in combination with a liposome and protamine.

Nucleic acid molecule expression for use in RNAi gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types, such as tumor cells, can be used to direct the expression of a nucleic acid. The enhancers used can include, without limitation, those that are characterized as tissue- or cell-specific enhancers.

### Combination Therapies

One or more HIPK1, HIPK2, or USP32 nucleic acids may be administered alone or in combination with any other standard proliferative disease therapy; such methods are known to the skilled artisan (e.g., Wadler et al., Cancer Res. 50:3473-86, 1990), and include, but are not limited to, chemotherapy, hormone therapy, immunotherapy, radiotherapy, and any other therapeutic method used for the treatment of a proliferative disease.

### III. Experimental Results

Experimental results demonstrating that HIPK1, HIPK2, and USP32 are essential for the growth and survival of neoplastic cells but not normal cells are set forth below.

### Screen for Modulators of Mitosis

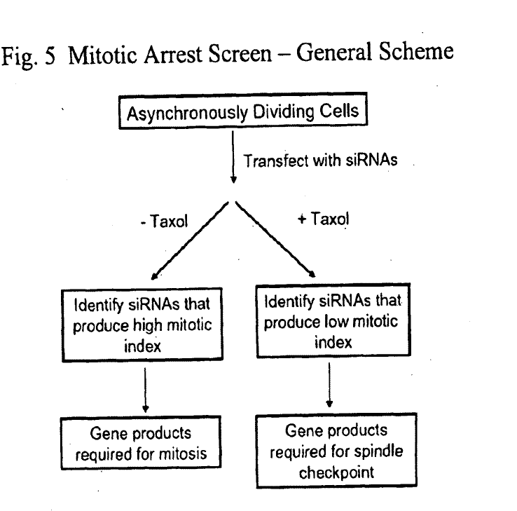

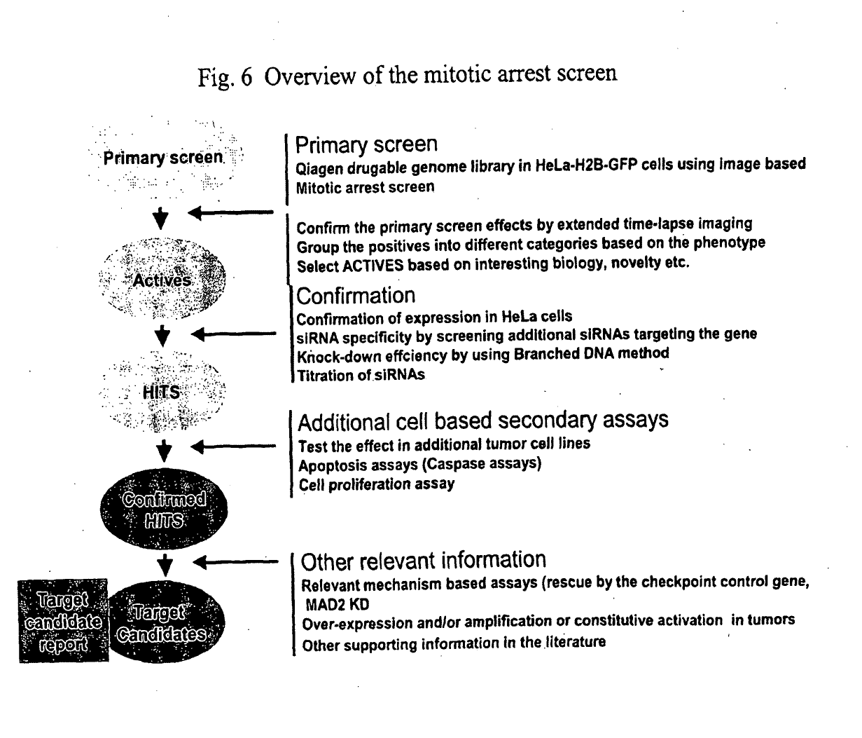

Two related screens have been utilized to identify gene products that positively or negatively regulate progression through mitosis (FIGS. 5 and 6). In the first screen, siRNAs that produce a mitotic arrest were identified. The increased mitotic index may result from inactivation of gene products that regulate mitotic progression, such as the Anaphase-Promoting Complex or Polo-like kinase. Alternatively, the mitotic arrest might occur indirectly, as a result of perturbation of a mechanical component of the mitotic spindle, such as the mitotic motor protein Eg5. In this case, the mitotic arrest is a secondary consequence of activation of the spindle checkpoint pathway, which delays progression through mitosis until chromosomes have become properly attached to the mitotic spindle. Treatment of cells with taxanes or the Eg5 inhibitor monastrol induces a checkpoint-dependent mitotic arrest. This screen identified regulators of mitosis and structural components of the mitotic spindle required for normal mitotic progression.

In the second screen, siRNAs required for spindle checkpoint function were identified. The spindle checkpoint pathway is important for accurate chromosome segregation in human cells. This pathway monitors attachment and tension of kinetochore-microtubule interactions, and inhibits anaphase onset until all kinetochores have become properly aligned at the metaphase plate. Many tumors exhibit defects in mitosis, including elevated rates of chromosome missegregation and multipolar spindle formation. In most cases, the specific molecular defects responsible for these abnormalities remain uncharacterized. However, it has recently been shown that dysregulation of the Rb/E2F pathway may lead to upregulation of expression of spindle checkpoint components, resulting in aberrant regulation of the checkpoint in human tumors. In other cases, mitotic abnormalities may lead to an increased requirement for checkpoint signaling for cancer cells to be able to segregate chromosomes. If so, then partial inhibition of spindle checkpoint signaling may lead to selective lethality in cancer cells.

In this assay, cells were transfected with siRNAs and then treated with taxol to induce a spindle-checkpoint dependent mitotic arrest. siRNAs that reduce the mitotic index were identified. If an siRNA inactivates the checkpoint, cells exit mitosis and return to interphase. However, the nuclei are generally abnormal in structure due to the aberrant exit from mitosis. The aberrant nuclear structure serves as a morphological “signature” of the perturbation of the pathway. This screen also identified siRNAs that arrest cells in interphase, prior to mitosis. In this case, the cells arrest in interphase, but the nuclei have a normal morphology, as they arrest prior to the point at which taxol acts. Thus this screening method has identified siRNAs that target the specific pathways of interest, and siRNAs that inhibit cellular proliferation through other mechanisms.

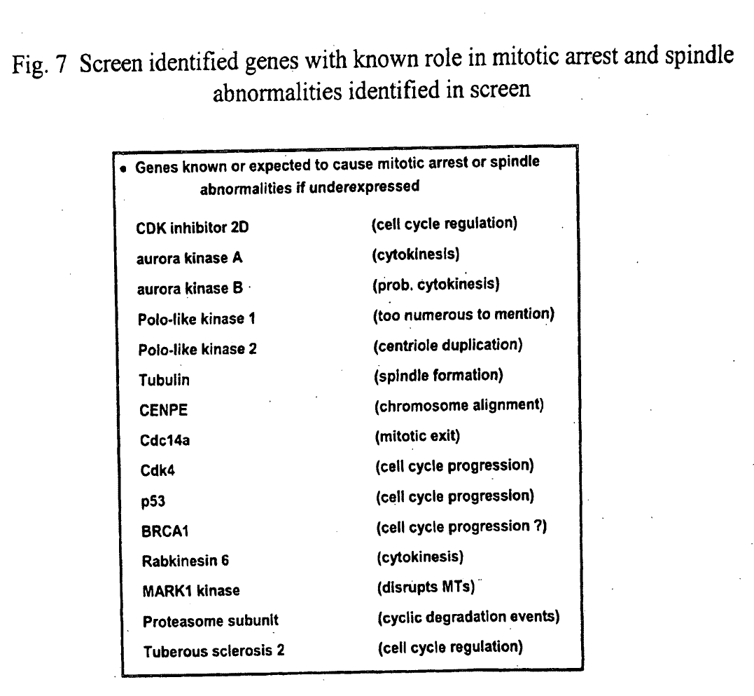

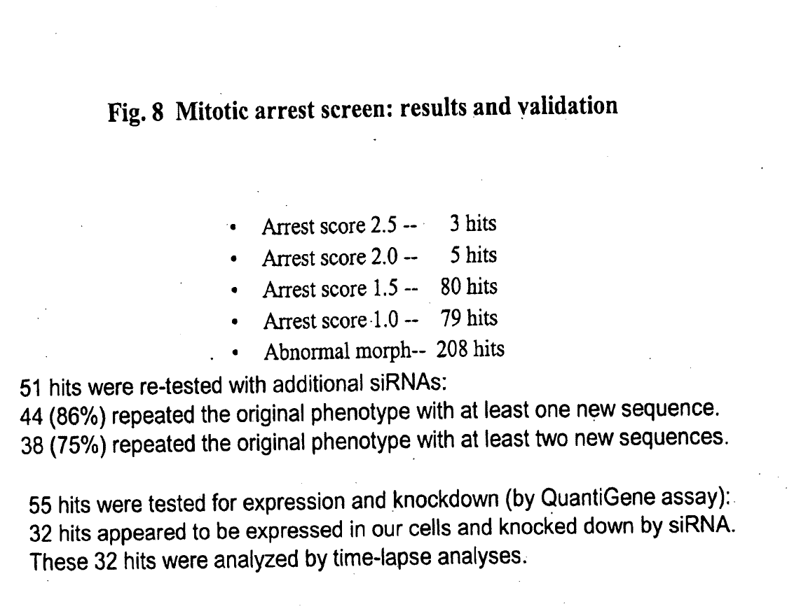

The entire set of 10,000 siRNAs have been screened in both assays (representing a total of 60 384-well plates). The screen identified Polo-like kinase, a known mitotic regulator, as a strong hit in the screen, thus validating the approach. Additional genes identified in this screen that were known to be involved in mitosis are set forth in FIG. 7. 375 siRNA constructs were identified which resulted in mitotic arrest (FIG. 8).

In order to confirm that the genes identified in the above screens were involved in mitosis, experiments were performed with additional siRNA constructs (FIG. 6). Furthermore, the relationship between the degree of decreased target gene expression and mitotic phenotype was examined with the Quantigene Assay (Genospectra Inc.). Target genes were further validated, where possible, with the degree of knockdown was correlated with an expression profile produced in HeLa cells in response to the anti-topoisomerase compound camptothecin (Carson et al. Cancer Res. 64: 2096-2104, 2004).

### Methods

The Qiagen druggable genome siRNA library (10,000 siRNAs targeting 5,000 genes) was reformatted into 30 384-well plates. For both screens, cells were transfected with siRNAs and then fixed and stained with DAPI (to stain DNA) and fluorescent phalloidin (to stain the actin cytoskeleton). High-throughput automated microscopy was used to identify siRNAs that increase the mitotic index (in the absence of taxol), or that decrease the mitotic index (in the presence of taxol).

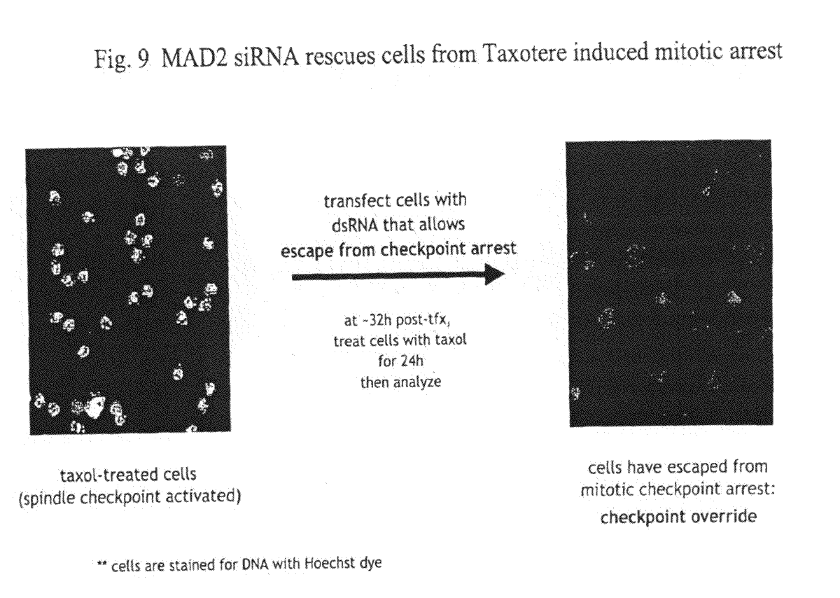

The positive control for the screens conducted in the absence of taxol contained an siRNA that targets the kinetochore protein Hec1 which induces a strong mitotic arrest in cells. This positive control was performed in each of the screening plates to measure transfection efficiency. In the screens conducted in the presence of taxol, cells, in the absence of siRNA, arrest in mitosis due to the taxol-dependent activation of the spindle checkpoint. Transfection of an siRNA that targets a known spindle checkpoint protein, Mad2, leads to reversal of the mitotic arrest, so that the cells accumulate in interphase (FIG. 9). Again, this positive control was included in each screening plate to monitor adequacy of transfection.

Approximately 16 hours prior to transfection, cells were plated in OptiMem media plus 1% fetal calf serum (FCS), in the presence of penicillin, streptomycin, and glutamate onto 384-well plates (black-side, clear bottom; Corning-Costar 3712). HeLa-H2B cells were plated at 10,000 cells per well (20 μl total volume per well) the night before the transfection. This usually yielded approximately 20-30% confluence by the next morning (the confluency is deliberately low for transfection because it is better for subsequent image-processing steps).

Approximately one to four hours prior to transfection 30 μl OptiMem medium without FCS was added to existing media in each well. Wells were aspirated, and 35 μl of the previously described OptiMem medium (with antibiotics, without FCS) was added and the cells were returned to incubator.

For fixation and staining, 2× fix/stain cocktail was added at an equal volume to the media in well. The 2× fix/stain contains 2 ml 37% formaldehyde, 2 μl tritc-phalloidin, 0.2 mg/ml in DMSO (Sigma P-1951), 0.5 μl Hoechst 33342 stain (Molecular Probes H-3570), 200 μl 10% Triton X-100, and 7.8 ml phospho-buffered saline (PBS). The cells were then incubated at room temperature for 20 minutes and washed two to three times with PBS.

Some versions of the experiment were automated. Again the final transfection volume was 40 μl. Each well contained 8 μl OptiMem, 0.5 μl GTS diluent, and 0.25 μl GeneSilencer. A 384-well source plate (AbGene AB-1055) with 8.8 μl of transfection cocktail per plate and about 10 μl of extra volume was added to allow for pipeting losses. Approximately 4 to 6 hours post-transfection, 20 μl of DMEM with 30% FCS and antibiotics were added. Cells were incubated for 48 hours post-transfection for phenotype development before processing. For the plus taxol screen, taxol was added to a 100-150 nM final concentration at approximately 32 to 36 hours post-transfection and then fixed 24 hours after taxol addition.

One feature of the invention is the use of time-lapse videomicroscopy. This custom-built microscope enables a person skilled in the art to simultaneously image cells at multiple positions on a cover slip or multi-well chamber for periods as long as a week. The microscope consists of a Nikon TE2000 inverted microscope, housed in a custom-designed incubator. This microscope allows measurement, on a cell-by-cell basis, of how a population of cells responds to a perturbation such as siRNA or drug treatment. Cells were imaged that express histone 2B fused to GFP, which allows monitoring of nuclear morphology during interphase and chromosome dynamics during mitosis. This enables determination as to whether cells proceed through mitosis normally or abnormally, and whether cells undergo apoptosis. In each experiment, typically 100 cells per field were imaged, and up to 40 fields were simultaneously acquired, providing information on the behavior of up to 4000 cells per experiment.

After images were acquired using the automated microscope, they were processed and measured to determine the mitotic index. Additional targets can be scored using one of the exemplified targets as a control. Additionally, each image was inspected manually to confirm the results of the automated measurement, and to identify other features of the image that might not be detected by the computer. For example, siRNAs have been identified that induce spindle abnormalities during mitosis without increasing the mitotic index.

### HIPK1, HIPK2, and USP32

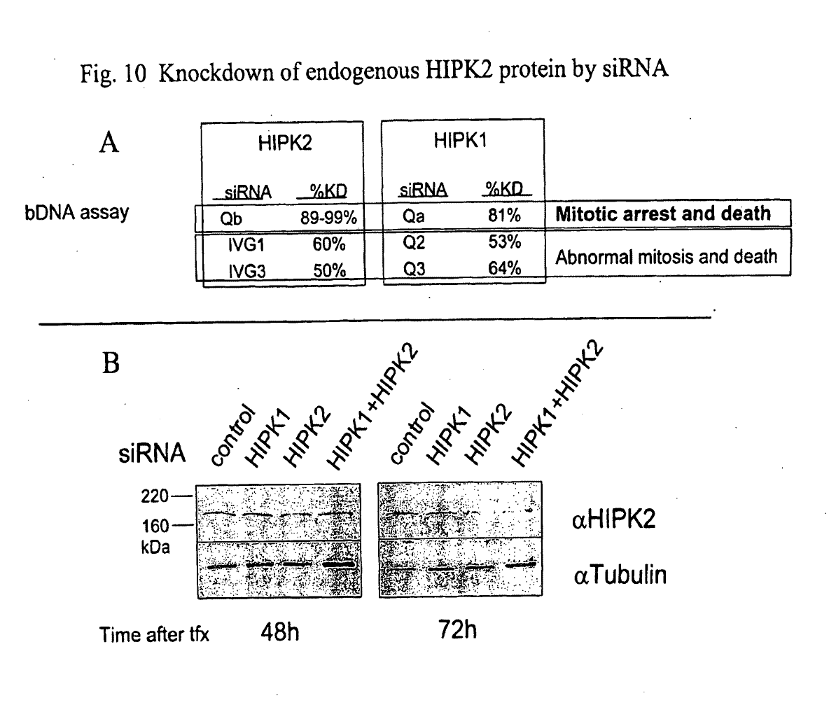

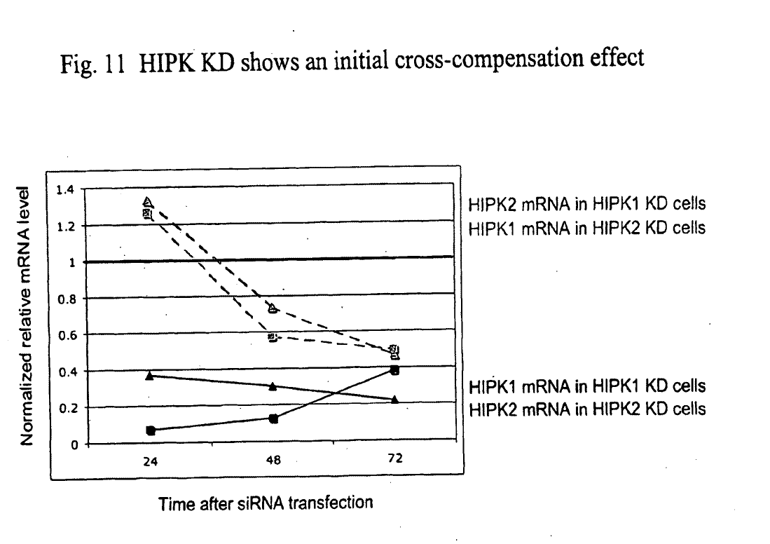

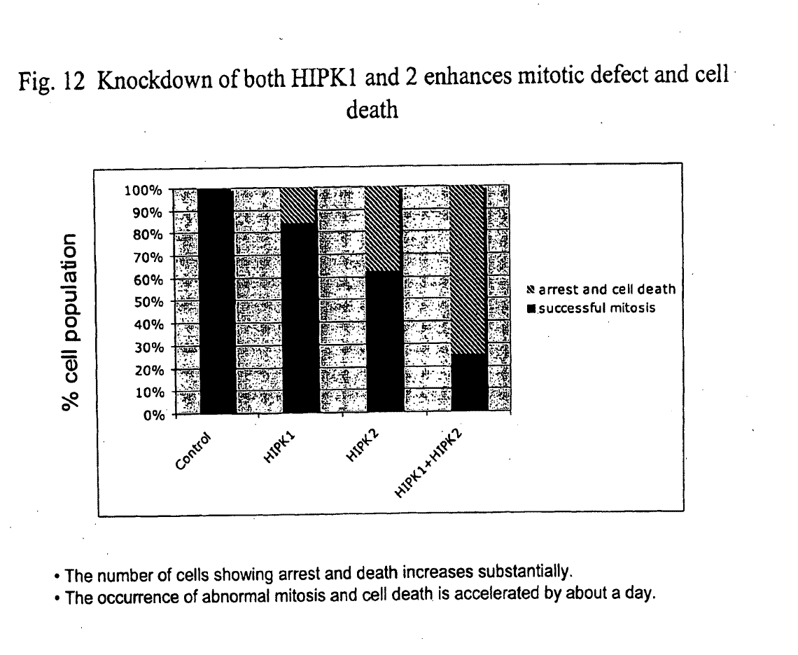

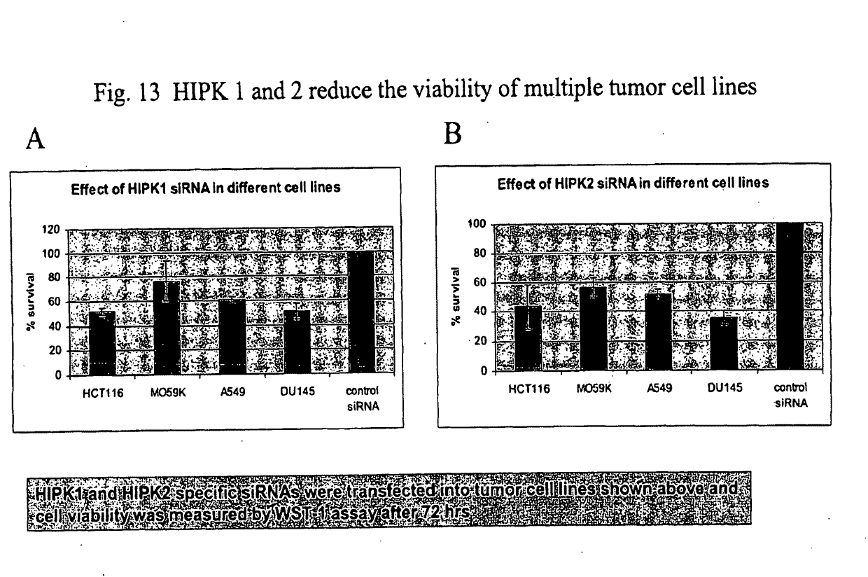

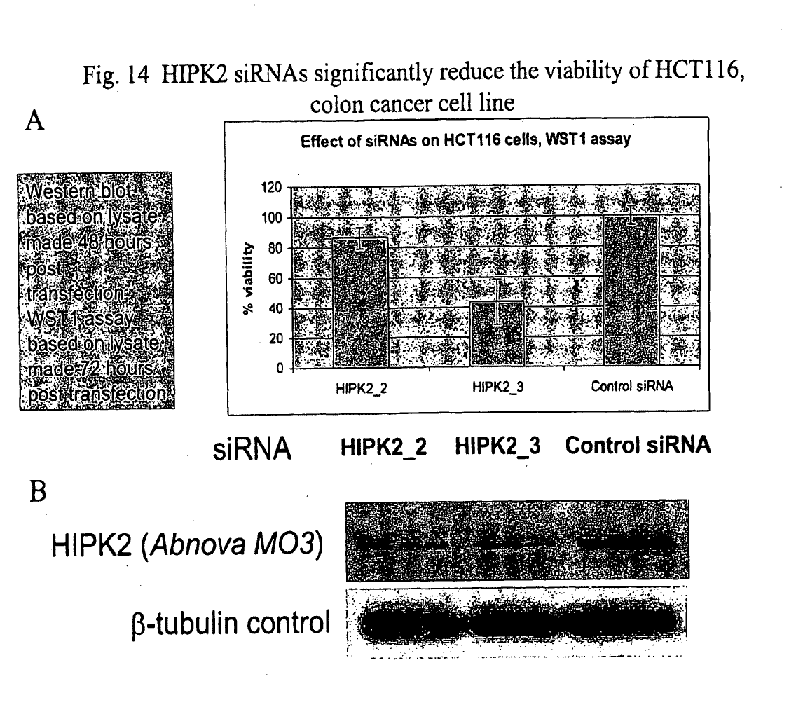

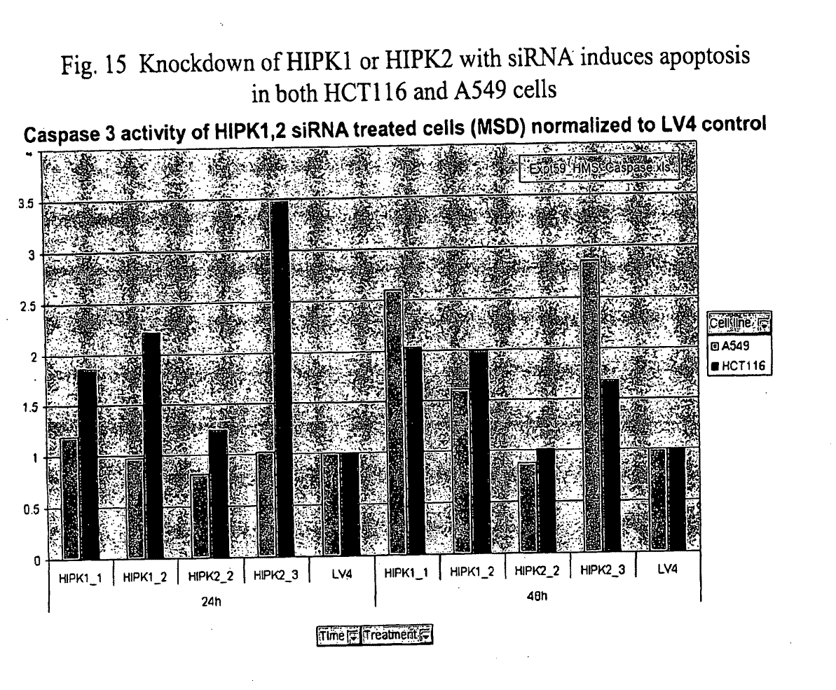

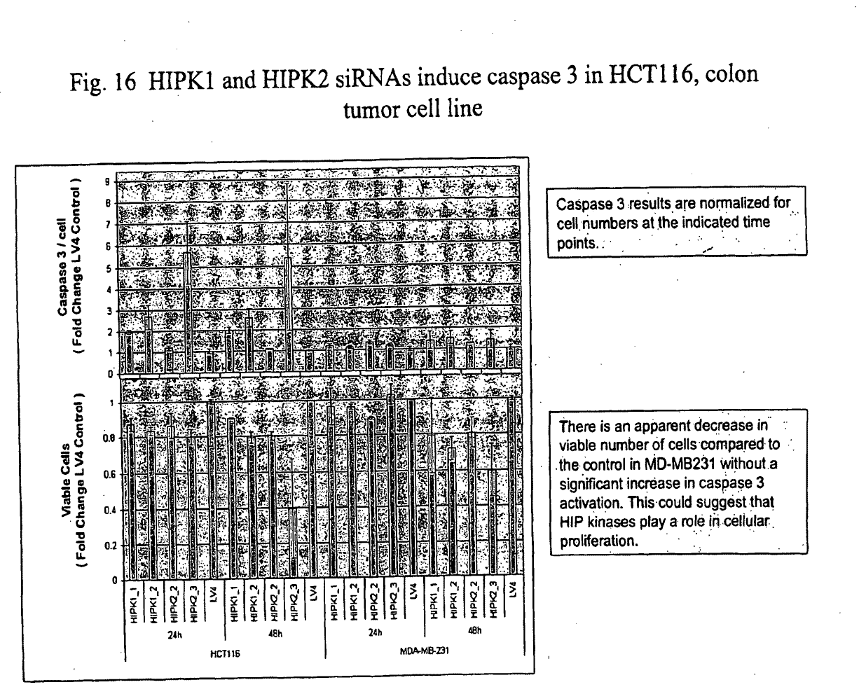

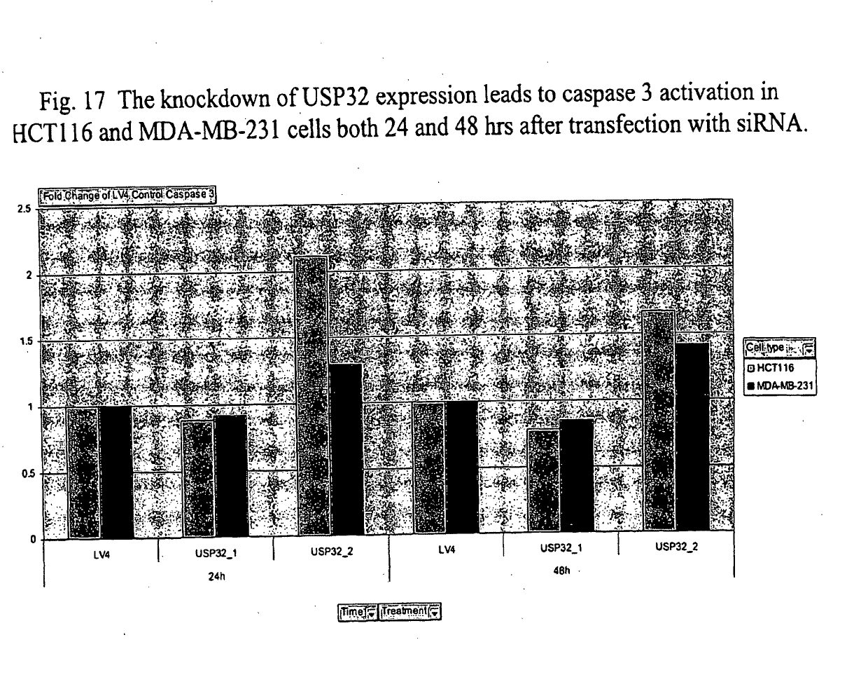

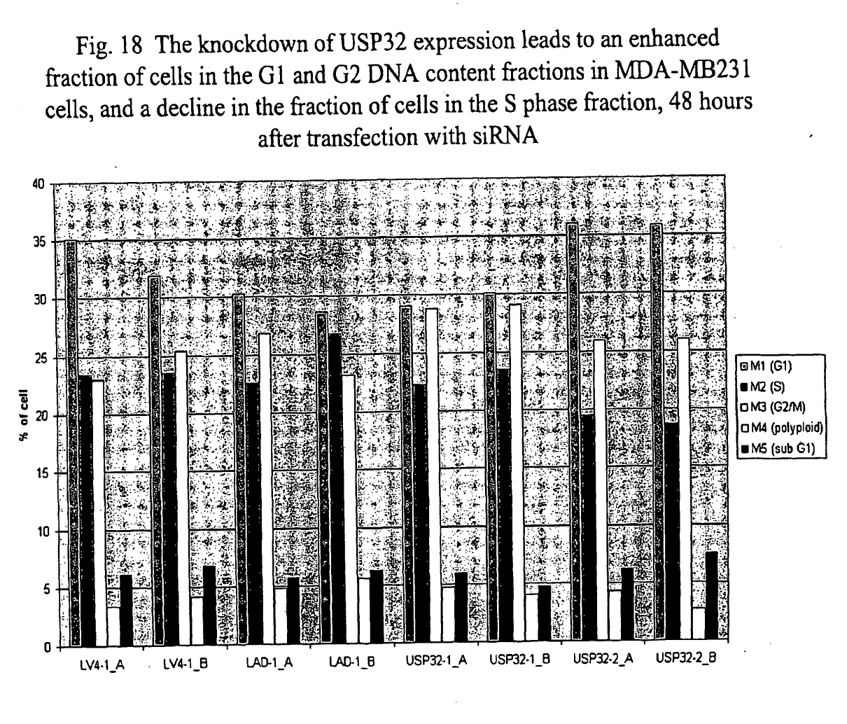

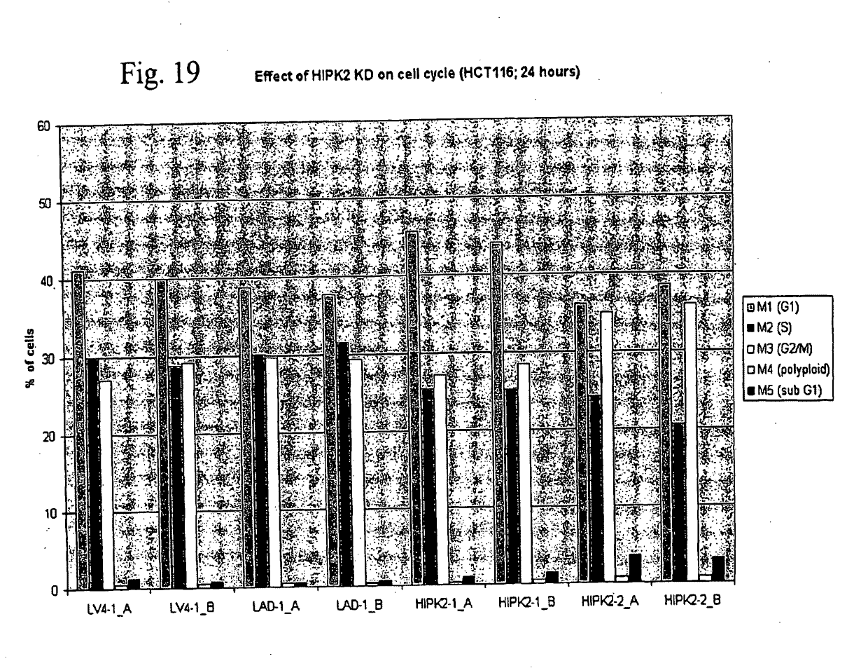

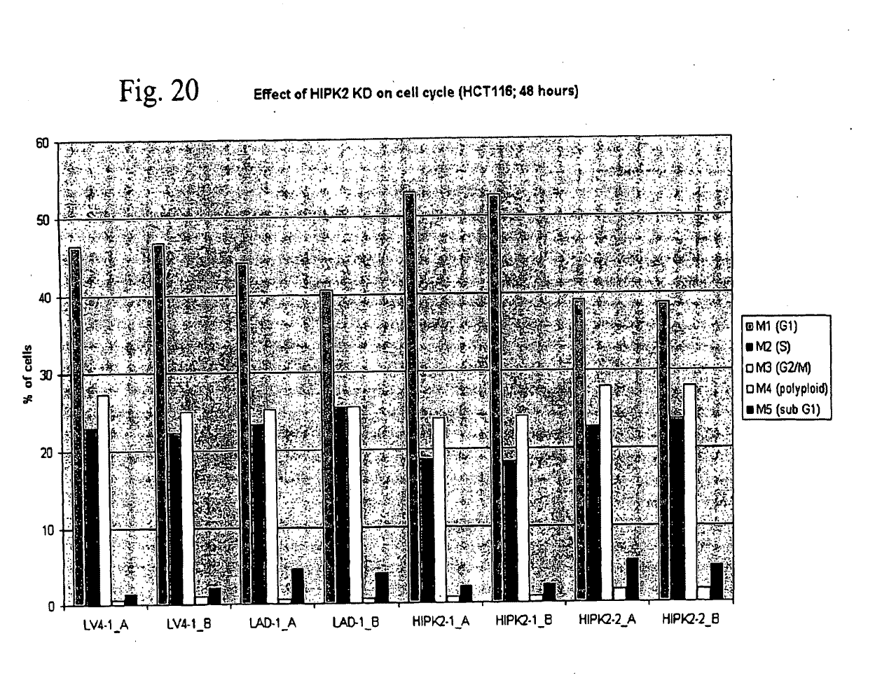

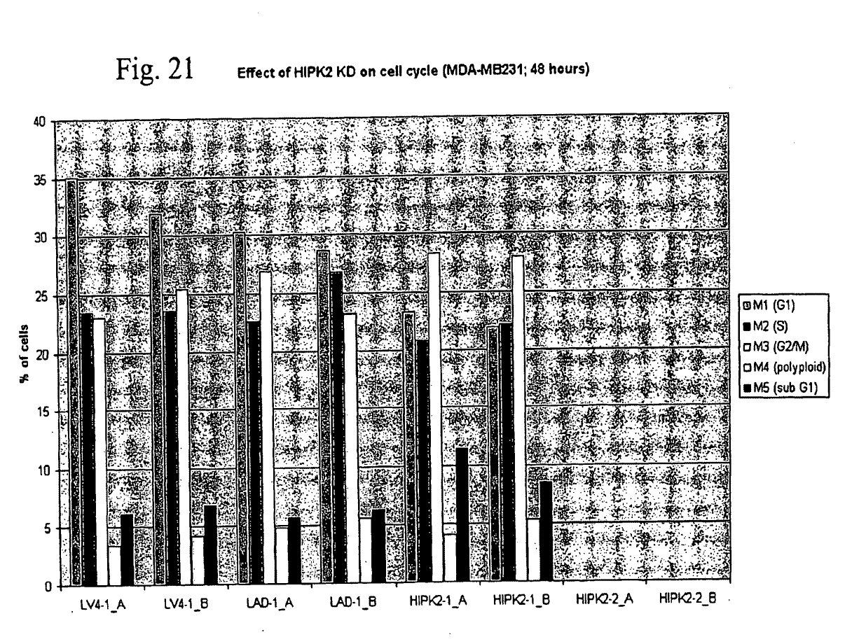

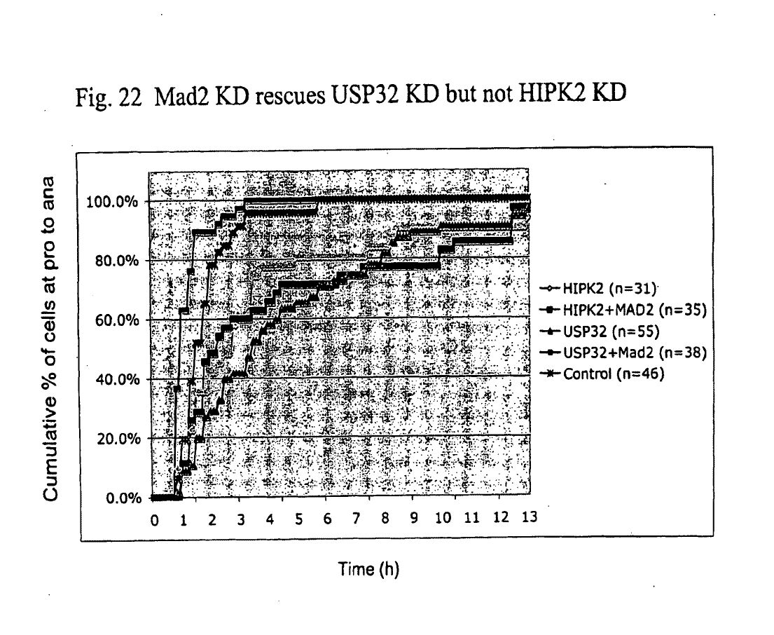

Using this screen, we identified HIPK1, HIPK2, and USP32 as being essential for mitosis in cells derived from neoplastic tissue. Protein and mRNA expression of HIPK1, HIPK2, and USP32 was decreased in cells treated with siRNA constructs to each of these genes (e.g., FIGS. 10 and 11). Cell survival was decreased in cells treated with HIPK1, HIPK2, and USP32 siRNA (FIGS. 12-14). This decrease in cell survival was associated with increased caspase 3 activity, an indicator of increased apoptosis (FIGS. 15-17). Treatment with HIPK1, HIPK2, and USP32 siRNA constructs resulted in changes in cell cycle distribution (FIGS. 18-22).

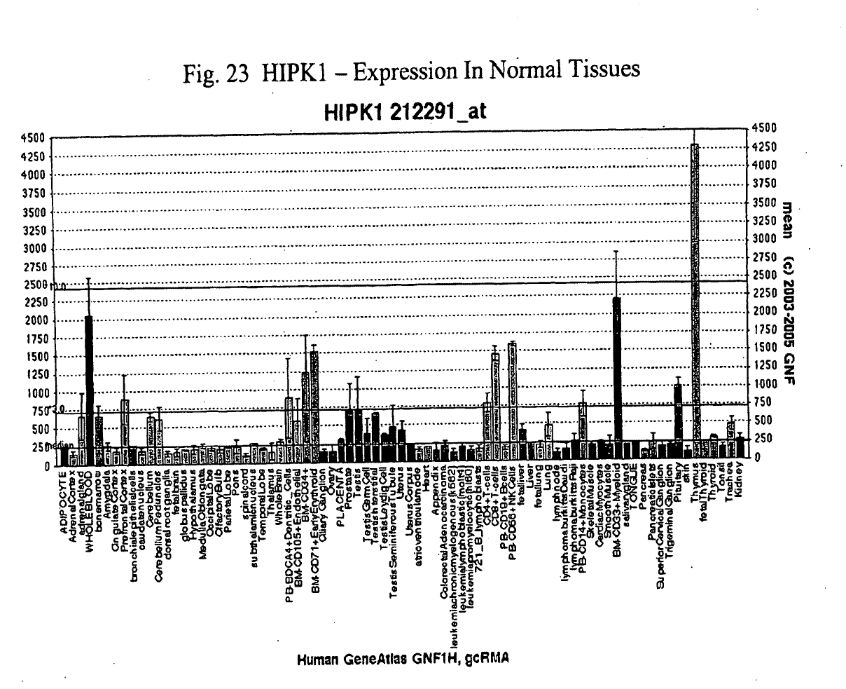

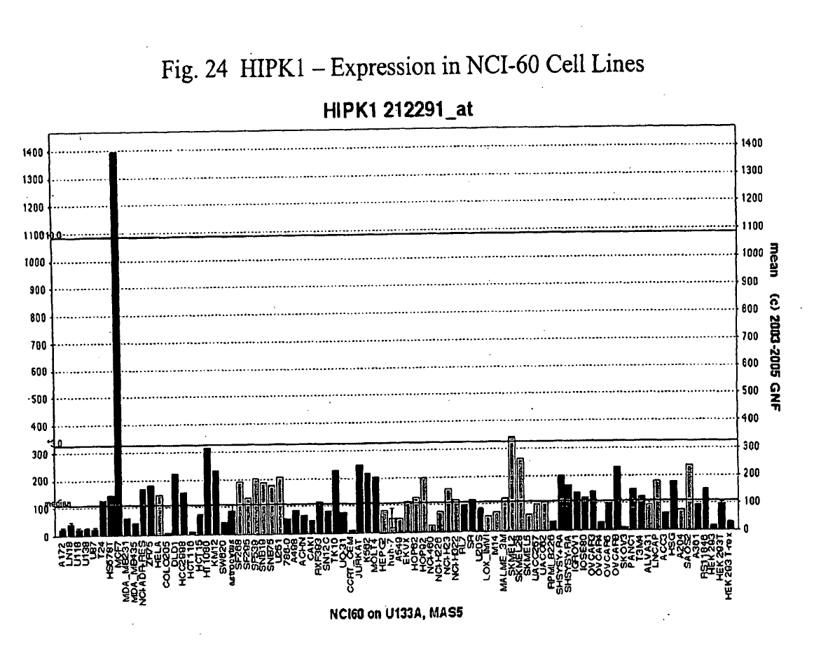

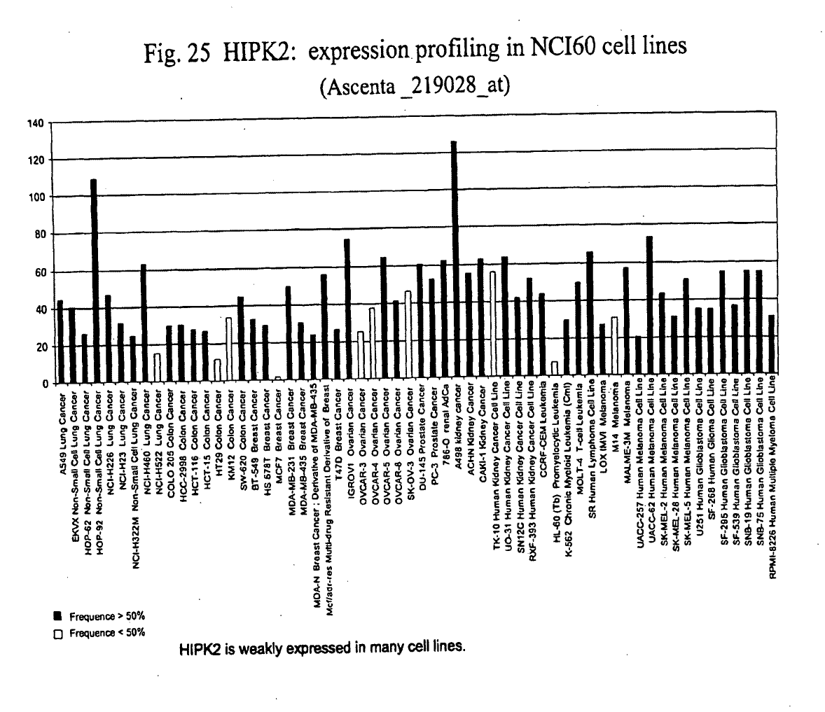

Further validating HIPK1 and HIPK2 as targets for anti-cancer therapy, expression of HIPK1 and HIPK2 is elevated in various cell lines derived from neoplastic tissue (FIGS. 23-25).

### Methods

### Cell Lines

Human cell lines HCT116 (colorectal carcinoma), M059K (glioblastoma), A549 (lung carcinoma), DU 145 (prostate carcinoma), and MDA-MB-231 (breast epithelial adenocarcinoma) were acquired from the American Type Culture Collection and maintained as suggested by the distributor.

siRNA Sequences

The following siRNA sequences were used to inhibit expression of HIPK1:

| | | |
|---|---|---|
| H1-1: | aaGGCTTGCCAGCTGAATATC | (Seq ID NO: 4) |
| H1-2: | agGGAAGCTGTACACCACTAA | (SEQ ID NO: 5) |

The following siRNA sequences were used to inhibit expression of HIPK2:

| | | |
|---|---|---|
| H2-1: | tcCCGAAGTCTCCATACTAAA | (SEQ ID NO: 6) |
| H2-3: | aaGGGTTTGCCTGCTGAATAT | (SEQ ID NO: 7) |

The following siRNA sequences were used to inhibit expression of USP32:

| | | |
|---|---|---|
| U1: | aaGCCTCAGTTACGTGAATAC | (SEQ ID NO: 8) |
| U2: | CCAGTAAAGGCTACATCAT | (SEQ ID NO: 9) |

The following siRNA sequence was used as a negative control:

| | | |
|---|---|---|
| LV4: | GUACGUUACGCGUAACGUAtt | (SEQ ID NO: 10) |

Transfection of siRNA

Cells were plated in 100 ul of medium the day before transfection. Lipofectamine 2000 was used to transfect siRNAs at 50 nM final concentration. Transfection mix was replaced with fresh media 24-48 hours later after transfection.

### Western Blotting to Detect HIPK2:

Cells were lysed in lysis buffer (50 mM Tris-HCl, pH 7.5; 100 mM NaCl; 1% NP-40; 2 mM EDTA; 1 mM sodium orthovanadate; 1 mM PMSF). Immediately before use, one Complete Mini EATA-free Protease Inhibitor cocktail tablet (Roche) was added per 2 mLs lysis buffer.

Cells were lysed in the aforementioned lysis buffer for 30 minutes on ice and then spun for 15 minutes at 14,000 RPM at 4° C. to eliminate cell debris. The supernatant was denatured with 4× NuPAGE LDS Sample Buffer (Invitrogen) at 75° C. for 10 minutes and fractionated by electrophoresis (20-50 μg of total protein per well) on a NuPAGE Bis-Tris gradient gel (412%, Invitrogen). Proteins were transferred onto nitrocellulose membrane (0.45 μm, Invitrogen). To block non-specific binding, the membranes were incubated in blocking buffer (TBS containing 5% non-fat dry milk and 0.1% Tween-20) at room temperature for 30 minutes, and incubated with monoclonal antibody to HIPK2 (Abnova H00028996-M03) overnight at 4° C. Membranes were incubated with horseradish peroxidase-conjugated secondary antibody in the blocking buffer for 2 hours at room temperature. After washing, the immune complexes were visualized by using the Enhanced Chemiluminescence System (Amersham, Buckinghamshire, UK).

### WST-1 Assays

Cells were transfected with siRNA, and media was changed 48 hours after transfection. After an additional 24 hours of incubation with fresh media, 10 μl of WST1 reagent was added to each well. Cells were incubated at 37° C. for 1 hour, and read on a microplate reader at a wavelength of 440 nm. A reference sample was read at 690 nm n.

### Caspase3 Assays

Cells were seeded at 0.3-0.5×10E5 cells/well onto 24 well plates, and transfected with siRNA at a concentration of 20 nM. Twenty four and 48 hours post transfection, cells were lysed using manufacturer's specified lysis buffer (Meso Scale Discovery, LLC). Lysates were analyzed using the caspase-3 assay from Meso Scale Discovery, and the assays were read using a MSD Sector Imager 2400.

### FACS Assays for Distribution of Cells in the Cell Cycle

At 24 and 48 hours after transfection of cells with siRNA, the floating cell population was removed and retained. Remaining adherent cells were trypsinized, and trypsin activity was stopped by addition of media. Trypsinized cells were pooled with the retained floating cell sub-population in a polycarbonate FACS compatible test-tube and pelleted, and the supernatant was decanted. Cells were fixed by serial addition of 0.5 mls cold PBS and (while vortexing) 2 mls ice cold 95% ethanol. Fixed cells were stained with propidium iodide and analyzed on a BD FACSCalibur Flow Cytometer. Data were analyzed using standard methods.

### OTHER EMBODIMENTS

The description of the specific embodiments of the invention is presented for the purposes of illustration. It is not intended to be exhaustive or to limit the scope of the invention to the specific forms described herein. Although the invention has been described with reference to several embodiments, it will be understood by one of ordinary skill in the art that various modifications can be made without departing from the spirit and the scope of the invention, as set forth in the claims. All patents, patent applications, and publications referenced herein are hereby incorporated by reference.

What is claimed is:

1. A method for identifying a candidate therapeutic compound comprising the steps of: (a) contacting HIPK1 with the compound to be assessed;
(b) comparing the biological activity of said HIPK1 with the biological activity of said HIPK1 absent said compound, and (c) identifying a compound which reduces the biological activity of said HIPK1; wherein said compound which reduces the biological activity of said HIPK1 is a candidate compound for the treatment of a proliferative disease.

2. The method according to claim 1, wherein said contacting is in a cell-free mixture.

3. The method according to claim 1, wherein said contacting is in a cell-based mixture.

4. The method according to claim 1, wherein said contacting is in a recombinant cell.

5. The method according to claim 1, wherein said biological activity of said HIPK1 is kinase activity.

6. The method according to claim 1, wherein said biological activity of said HIPK1 is binding activity.

7. The method according to claim 1, wherein said proliferative disease is selected from a group consisting of: colon cancer, breast cancer, prostate cancer, lymphoma, leukemia, melanoma, ovarian cancer, pancreatic cancer, and lung cancer.

8. A method for identifying a candidate therapeutic compound comprising the steps of: (a) contacting HIPK2 with the compound to be assessed; (b) comparing the biological activity of said HIPK2 with the biological activity of said HIPK2 absent said compound, and (c) identifying a compound which reduces the biological activity of said HIPK2; wherein said compound which reduces the biological activity of said HIPK2 is a candidate compound for the treatment of a proliferative disease.

9. The method according to claim 8, wherein said contacting is in a cell-free mixture.

10. The method according to claim 8, wherein said contacting is in a cell-based mixture.

11. The method according to claim 8, wherein said contacting is in a recombinant cell.

12. The method according to claim 8, wherein said biological activity of said HIPK2 is kinase activity.

13. The method according to claim 8, wherein said biological activity of said HIPK2 is binding activity.

14. The method according to claim 8, wherein said proliferative disease is selected from a group consisting of: colon cancer, breast cancer, prostate cancer, lymphoma, leukemia, melanoma, ovarian cancer, pancreatic cancer, and lung cancer.

15. A method for identifying a candidate therapeutic compound comprising the steps of: (a) contacting USP32 with the compound to be assessed; (b) comparing the biological activity of said USP32 with the biological activity of said USP32 absent said compound, and (c) identifying a compound which reduces the biological activity of said USP32; wherein said compound which reduces the biological activity of said USP32 is a candidate compound for the treatment of a proliferative disease.

16. The method according to claim 15, wherein said contacting is in a cell-free mixture.

17. The method according to claim 15, wherein said contacting is in a cell-based mixture.

18. The method according to claim 15, wherein said contacting is in a recombinant cell.

19. The method according to claim 15, wherein said biological activity of said USP32 is protease activity.

20. The method according to claim 15, wherein said biological activity of said USP32 is binding activity.

21. The method according to claim 15, wherein said proliferative disease is selected from a group consisting of: colon cancer, breast cancer, prostate cancer, lymphoma, leukemia, melanoma, ovarian cancer, pancreatic cancer, and lung cancer.

22. A method of identifying a candidate compound that ameliorates a proliferative disease, said method comprising the steps of (a) contacting a cell with a candidate compound, (b) comparing the expression of at least one of HIPK1, HIPK2, and USP32 with the expression of said gene or genes in a cell not contacted with said candidate compound, and (c) identifying a compound which modulates the expression of at least one of said genes.

23. The method of claim 22, wherein said expression is assessed by reduction in disease-specific properties.

24. The method of claim 22, wherein said cell is contained within an animal model.

25. The method of claim 22, wherein said cell is derived from a disease model.

26. The method of claim 22 wherein said expression is measured using a microarray.

27. The method of claim 26, wherein said microarray is a nucleic acid microarray.

28. The method of claim 26 wherein said microarray is a protein microarray.

29. A method of treating a proliferative disease in a subject comprising administering an inhibitor of HIPK1 to said subject, wherein said inhibitor of HIPK1 is an siRNA construct.

30. A method of treating a proliferative disease in a subject comprising administering an inhibitor of HIPK2 to said subject, wherein said inhibitor of HIPK2 is an siRNA construct.

31. A method of treating a proliferative disease in a subject comprising administering an inhibitor of USP32 to said subject, wherein said inhibitor of USP32 is an siRNA construct.
